(19) United States
(12) Patent Application Publication
(10) Pub. No.: US 2023/0165943 A1
(43) Pub. Date: Jun. 1, 2023

(54) TP508 ACUTE THERAPY FOR PATIENTS WITH RESPIRATORY VIRUS INFECTION

(71) Applicants: Darrell CARNEY, Galveston, TX (US); Laurie E. SOWER, Galveston, TX (US); Chrysalis BioTherapeutics, Galveston, TX (US)

(72) Inventors: LAURIE E. SOWER, Galveston, TX (US); DARRELL CARNEY, Galveston, TX (US)

(21) Appl. No.: 17/917,754

(22) PCT Filed: Apr. 7, 2021
(86) PCT No.: PCT/US21/26257

§ 371 Date: Oct. 7, 2022

Related U.S. Application Data
(60) Provisional application No. 63/006,492, filed on Apr. 7, 2020.

(51) Int. Cl. A61K 38/48; A61P 11/00; A61P 31/14; A61P 29/00

(52) U.S. Cl. CPC A61K 38/4833; A61P 11/00; A61P 31/14; A61P 29/00; C12Y 304/21005

(57) ABSTRACT

Certain embodiments are directed to using TP508 as a drug that can restore prevent alveolar damage, reducing pulmonary edema and prevent respiratory failure and mortality-associated progression of COVID-19.

### PRIORITY PARAGRAPH

This Application claims priority to U.S. Provisional Patent Application Ser. No. 63/006,492 filed Apr. 7, 2020 which is incorporated herein by reference in its entirety.

### BACKGROUND

A respiratory disease (Coronavirus Disease 2019 (COVID-19)) caused by a novel coronavirus (SARS-CoV-2) was detected in China and has now been detected internationally, including detection in the United States. On Jan. 30, 2020, the International Health Regulations Emergency Committee of the World Health Organization declared the outbreak a public health emergency of international concern (PHEIC). On Jan. 31, 2020 a public health emergency (PHE) was declared for the United States.

Although approximately 80% of SARS-CoV-2-infected people have mild to moderate flu-like symptoms, about 15% develop severe pneumonia with shortness of breath, and about 5% reach a critical stage with respiratory failure, septic shock and potential for multi-organ failure. An early report of Chinese COVID-19 outcomes showed that fatality was primarily observed in elderly (15% mortality in >80 years-old; 8% in those between 70 and 79 years-old). As of February 29th, the 80+ year-old death rate was 21.9% of confirmed cases (World Health Organization-China Mission). The Chinese report also indicated that viral infection was lethal in 49% of critical stage elderly patients if they had preexisting hypertension, diabetes, cardiovascular or chronic pulmonary disease. All of these comorbidities are associated with vascular changes, endothelial dysfunction, loss of nitric oxide signaling, and a strong inflammatory cytokine production. Studies show that endothelial dysfunction and loss of nitric oxide production in lung endothelial cells causes increased vascular permeability, alveolar edema, and infiltration of inflammatory cells in alveolar spaces. In patients with ARDS, the loss of endothelial function, edema, inflammatory cell infiltration, and coagulopathies are related to disease progression and respiratory failure. The pathology of COVID-19 progression in the lung is similar to that seen in ARDS patients.

There is a need for improved methods for treating viral infections and the sequalae of viral infections, in particular coronavirus infection.

### SUMMARY

The SARS-CoV-2 pandemic (COVID-19) and the lethality of the virus, especially in senior citizens and those with preexisting vascular conditions, has accelerated clinical testing of various antiviral and immune modulating drugs. The underlying epithelial and vascular conditions that are causally linked to the severity of the infection and mortality, however, have not yet been fully addressed. There remains an urgent clinical need for supportive care drugs that when used with standard therapy would help to resolve Acute Respiratory Distress Syndrome (ARDS) and prevent death caused by SARS-CoV-2 infection. TP508, an active peptide of thrombin, can be used to address ARDS and post-respiratory viral syndrome. TP508 is a drug that can restore endothelial function, modulate inflammation, and prevent alveolar damage, reducing pulmonary edema and prevent respiratory failure and mortality-associated progression of COVID-19.

Certain embodiments are directed to methods of treating a subject with respiratory distress or a subject at risk of developing respiratory distress comprising administering to the subject a thrombin peptide derivative. In certain aspects the thrombin peptide derivative is TP508 or a derivate thereof. In certain aspects, the subject is experiencing acute respiratory distress (ARDS). In certain aspects, the formulation is administered to the subject by injection. In particular aspects, the formulation is administered to the subject by intravenous (IV) injection. In other aspects, the formulation is administered to the subject by subcutaneous injection. In still other aspects, the formulation is administered to the subject by inhalation or instillation. The subject can be diagnosed with a viral infection or other condition that puts the subject at risk for the development of ARDS. In certain aspects, the subject is diagnosed with a respiratory virus infection. In particular aspects, the subject is diagnosed with a coronavirus or influenza infection. In certain aspects, the subject is diagnosed with or suspected to have or at risk of having a SARS-CoV-2 infection.

For some patients who have had a respiratory infection, e.g., COVID-19, symptoms of the disease may last after the infection is over. These patients are “long haulers,” who have what is known as post-respiratory virus syndrome or post-COVID-19 syndrome, that may need extended treatment and rehabilitation to return to daily activities or work.

Post-infection syndrome can present as neurological sequalae (headaches, dizziness, weakness), fatigue (in particular a profound fatigue), and shortness of breath. A respiratory viral infection can cause long-term changes in the lungs and can lead to long-term dyspnea. Post-infection syndrome can occur in asymptomatic subjects, subjects with mild symptoms, intermediate symptoms, or severe symptoms. In certain aspects the post-infection syndrome worsens over time. In certain aspects about 1, 2, 3, or 4 weeks after infection, with no relationship with age. In certain aspects, post-infection syndrome is ameliorated by administering TP508 or a derivative thereof 1, 2, 3, 4, 5, 6, 7, 8, 9, 10, 11, 12, 13, 14, to 15 days or weeks (including all ranges and values there between, after infection, after a known exposure to the virus, or after the initiation of symptoms of infection. In certain aspects, TP508 is administered 5 to 15 days post infection. The term “infection” as used herein refers to the initial entry of a pathogen into a host; and the condition in which a pathogen has become established in or on cells or tissues of a host; such a condition does not necessarily constitute or lead to a disease. As it related to this embodiment of ameliorating post infection syndrome the time of infection can be determine by (i) detection of viral presence in the subject, (ii) exhibition of symptoms (e.g., loss of smell, loss of taste, elevated fever, and the like), or (iii) knowledge that the subject was in a location known for active transmission of the respiratory virus (this would also be taken as a subject at risk of infection). The term “respiratory virus” refers to a virus which infects cells of the respiratory tract, such as cells lining the oral cavity, nasopharynx, throat, larynx, bronchi and bronchioles, etc. Respiratory viruses include influenza virus, rhinovirus, adenovirus, respiratory syncytial virus (RSV), coronavirus, severe acute respiratory syndrome (SARS)-associated coronavirus, measles virus, mumps virus, parainfluenza virus, rubella virus, poxvirus, parvovirus, hantavirus and varicella virus. Statements and description which use the term “respiratory virus” indicate, and refer to, any one or more of the respiratory viruses listed herein unless the virus is specifically identified. “Exposure” to a virus denotes encounter with virus which allows infection, such as, for example, upon contact with an infected individual or virus containing droplets or aerosol. An individual who is “at risk of being exposed” to a virus is an individual who may encounter the virus such that the virus infects the individual (i.e., virus enters cells and replicates). In some contexts, an individual is determined to be “at risk” because exposure to the virus has higher probability of leading to infection (such as with immunocompromised and/or elderly) which can further result in serious symptoms, conditions, and/or complications. In some settings, including, but not limited to, institutions such as hospitals, schools, day care facilities, military facilities, nursing homes and convalescent homes, an individual is determined to be “at risk” because of time spent in close proximity to others who may be infected or have been identified as infected when in close proximity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Effect of TP508 on TNFα-induced loss of endothelial barrier function.

FIG. 2. Gene array analysis of human coronary artery endothelial cells (HCAE cells) 6 h after treatment with TNFα or TNFα+TP508.

FIG. 3. TGFβ2 expression in human endothelial cells 6 h after treatment with: saline (C N 6 h); TNFα (TN 6 h); TNFα+TP508 (T+TP N 6 h); or TP508 alone (TP N 6 h).

FIG. 4. C5a-R1 expression in human endothelial cells 6 h after treatment with saline (C N 6 h); TNFα (T N 6 h); TNFα+TP508 (T+TP N 6 h); or TP508 alone (TP N 6 h).

FIG. 5. TP508 Effect on Cytokine Expression in Bronchial Lavage. (Left) Values represent the % of expression relative to that observed in CoV-2 challenged controls with a single SC injection of 25 mg/kg TP508 24 h after infection with SARS-CoV-2. (Right) Values represent the % of expression relative to that observed in CoV-2 challenged controls with a single SC injection of 25 mg/kg TP508 24 h after infection with SARS-CoV-2.

FIG. 6. Effect of TP508 on Cigarette Smoke (CF) upregulation of GM-CSF compared to levels in filtered air.

FIG. 7. TP508 prevents lymphocyte infiltration through alveoli into lungs 24 h post LPS exposure.

### DESCRIPTION

The SARS-CoV-2 virus is a betacoronavirus, similar to MERS-CoV and SARS-CoV, that can result respiratory distress. TP508 is a drug that targets multiple cell types to mitigate vascular damage, modulate immune responses, and restore normal function to tissues following injury, ischemia, and radiation exposure. In vitro and in vivo studies have shown that TP508 reverses vascular endothelial dysfunction and restores nitric oxide (NO) signaling to protect vascular endothelial cells, mitigates tissue damage, and prevents increases in vascular permeability. TP508 is currently being developed as a post-radiation exposure, injectable drug, to mitigate effects of nuclear radiation exposure and to protect normal tissue from detrimental side effects of radiotherapy. TP508 (1) has shown efficacy in tissue regeneration clinical trials; (2) has an extensive nonclinical and clinical safety profile; (3) is currently being developed with BARDA as a medicinal countermeasure for acute radiation syndrome; (4) is GMP manufactured with sufficient inventory to enter clinical trials with COVID-19 patients given FDA's approval; (6) is easy to manufacture in large quantities; and (7) is cost effective.

### I. Thrombin Derivative Peptides and Thrombin Peptide TP508

TP508 represents a regenerative portion of human thrombin that is released from dissolving blood clots at sites of injury. This portion of thrombin stimulates regeneration of tissue. TP508 has been shown to: (i) stimulate revascularization and restoration of tissue repair in multiple tissues; (ii) protect, recruit, and stimulate proliferation of progenitor stem cells at sites of injury; (iii) modulate immune responses; (iv) restore nitric oxide (NO)-dependent endothelial function; (v) prevent apoptosis; and (vi) mitigate effects of radiation to prevent multiple organ failure and increase survival.

It has been discovered that TP508 prevents TNFα-induced permeability of human pulmonary endothelial cells in vitro. TP508 also counteracts the proinflammatory effects of TNFα on endothelial cells and monocytes, thus serving to reverse pathological inflammatory responses. Described below are methods and composition for the use of TP508 to reduce the progression of ARDS and mortality associated with COVID-19,

Thrombin peptide derivatives (also: “thrombin derivative peptides”) are analogs of thrombin that have an amino acid sequence derived at least in part from that of thrombin and are active at the non-proteolytically activated thrombin receptor (NPAR) Thrombin peptide derivatives can include, for example, peptides that are produced by recombinant DNA methods, peptides produced by enzymatic digestion of thrombin, and peptides produced synthetically, which can comprise amino acid substitutions compared to thrombin and/or modified amino acids, especially at one or both termini.

Thrombin peptide derivatives of the present invention include thrombin derivative peptides described in U.S. Pat. Nos. 5,352,664 and 5,500,412, each of which is incorporated herein by reference in their entirety. In one embodiment, the thrombin peptide derivatives of the present invention is a thrombin peptide derivative or a physiologically functional equivalent, i.e., a polypeptide with no more than about fifty amino acids, preferably no more than about thirty amino acids and having sufficient homology to the fragment of human thrombin corresponding to thrombin amino acids 508-530 (Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val; SEQ ID NO:1; TP508) that the polypeptide activates NPAR.

In another embodiment, the thrombin peptide derivatives of the present invention is a thrombin peptide derivative comprising a moiety represented by Structural Formula (I) Asp-Ala-R, where R is a serine esterase conserved domain. Serine esterases, e.g., trypsin, thrombin, chymotrypsin and the like, have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in aspects which do not affect the function of the thrombin receptor binding domain or the serine esterase conserved amino acid sequence. Such aspects may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

In one embodiment, the serine esterase conserved sequence comprises the amino acid sequence of SEQ ID NO:2 (Cys-Glu-Gly-Asp-Ser-Gly-Gly-Pro-Phe-Val) or a C-terminal truncated fragment of a polypeptide having the amino acid sequence of SEQ ID NO:2. It is understood, however, that zero, one, two or three amino acids in the serine esterase conserved sequence can differ from the corresponding amino acid in SEQ ID NO:2. Preferably, the amino acids in the serine esterase conserved sequence which differ from the corresponding amino acid in SEQ ID NO:2 are conservative substitutions as defined below, and are more preferably highly conservative substitutions. A “C-terminal truncated fragment” refers to a fragment remaining after removing an amino acid or block of amino acids from the C-terminus, said fragment having at least six and more preferably at least nine amino acids.

In another embodiment, the serine esterase conserved sequence comprises the amino acid sequence of SEQ ID NO:3 (Cys-X1-Gly-Asp-Ser-Gly-Gly-Pro-X2-Val; X1 is Glu or Gln and X2 is Phe, Met, Leu, His or Val) or a C-terminal truncated fragment thereof having at least six amino acids, preferably at least nine amino acids. In a preferred embodiment, the thrombin peptide derivative comprises a serine esterase conserved sequence and a polypeptide having a more specific thrombin amino acid sequence Arg-Gly-Asp-Ala. One example of a thrombin peptide derivative of this type comprises Arg-Gly-Asp-Ala-Cys-X1-Gly-Asp-Ser-Gly-Gly-Pro-X2-Val (SEQ ID NO:4). X1 and X2 are as defined above. The thrombin peptide derivative can comprise the amino acid sequence of Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:5) or an N-terminal truncated fragment thereof, provided that zero, one, two or three amino acids at positions 1-9 in the thrombin peptide derivative differ from the amino acid at the corresponding position of SEQ ID NO:4. Preferably, the amino acid residues in the thrombin peptide derivative which differ from the corresponding amino acid residues in SEQ ID NO:4 are conservative substitutions as defined below, and are more preferably highly conservative substitutions. An “N-terminal truncated fragment” refers to a fragment remaining after removing an amino acid or block of amino acids from the N-terminus, preferably a block of no more than six amino acids, more preferably a block of no more than three amino acids.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRaRb, wherein Ra and Rb are independently hydrogen, a C1-C10 substituted or unsubstituted aliphatic group, or Ra and Rb, taken together with the nitrogen to which they are bonded, form a C1-C10 non-aromatic heterocyclic group, and said N-terminal acyl group is represented by RcC(O)—, wherein Rc is hydrogen, a C1-C10 substituted or unsubstituted aromatic group, or a C1-C10 substituted or unsubstituted aromatic group. In another specific embodiment, the N-terminus of the thrombin peptide derivative is free (i.e., unsubstituted) and the C-terminus is free (i.e., unsubstituted) or amidated, preferably as a carboxamide (i.e., —C(O)NH₂). In a specific embodiment, the thrombin peptide derivative comprises the following amino acid sequence: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:5). In another specific embodiment, the thrombin peptide derivative comprises the amino sequence of Arg-Gly-Asp-Ala-Cys-Glu-Gly-Asp-Ser-Gly-Gly-Pro-Phe-Val (SEQ ID NO:6). Alternatively, the thrombin peptide derivative comprises the amino acid sequence of Asp-Asn-Met-Phe-Cys-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe (SEQ ID NO:7), The thrombin peptide derivatives can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acids at positions 1-9 and 14-23 in the thrombin peptide derivative can differ from the corresponding amino acids.

Preferably, the amino acids in the thrombin peptide derivative which differ from the corresponding amino acids are conservative substitutions as defined below, and are more preferably highly conservative substitutions. Alternatively, an N-terminal truncated fragment of the thrombin peptide derivative having at least fourteen amino acids or a C-terminal truncated fragment of the thrombin peptide derivative having at least eighteen amino acids can be used in the methods of the present invention.

A “C-terminal truncated fragment” refers to a fragment remaining after removing an amino acid or block of amino acids from the C-terminus. An “N-terminal truncated fragment” refers to a fragment remaining after removing an amino acid or block of amino acids from the N-terminus. It is to be understood that the terms “C-terminal truncated fragment” and “N-terminal truncated fragment” encompass acylation at the N-terminus and/or amidation at the C-terminus, as described above.

A preferred thrombin peptide derivative for use in the disclosed method comprises the amino acid sequence Ala-Gly-Tyr-Lys-Pro-Asp-Glu-GIy-Lys-Arg-Gly-Asp-Ala-Cys-X1-Gly-Asp-Ser-Gly-Gly-Pro-X2-Val (SEQ ID NO:8). Another preferred thrombin peptide derivative for use in the disclosed method comprises the amino acid sequence of Asp-Asn-Met-Phe-Cys-Ala-Gly-Tyr-Lys-Pro-Asp-Glu-Gly-Lys-Arg-Gly-Asp-Ala-Cys-X1-Gly-Asp-Ser-Gly-Gly-Pro-X2-Val-Met-Lys-Ser-Pro-Phe (SEQ ID NO:9), wherein X1 is Glu or Gln; X2 is Phe, Met, Leu, His or Val. The thrombin peptide derivatives can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acids, or C-terminal truncated fragments of these preferred thrombin peptide derivatives, the C-terminal truncated fragments having at least eighteen amino acids, can also be used in the disclosed method.

TP508 is an example of a thrombin peptide derivative and is 23 amino acid residues long, wherein the N-terminal amino acid residue Ala is unsubstituted and the COOH of the C-terminal amino acid Val is modified to an amide represented by —C(O)NH₂. Another example of a thrombin peptide derivative comprises the amino acid sequence where both N- and C-termini are unsubstituted (“deamide TP508”). Other examples of thrombin peptide derivatives which can be used in the disclosed method include N-terminal truncated fragments of TP508 (or deamide TP508), the N-terminal truncated fragments having at least fourteen amino acids, or C-terminal truncated fragments of TP508 (or deamide TP508), the C-terminal truncated fragments having at least eighteen amino acids.

As used herein, a “conservative substitution” in a polypeptide or peptide is the replacement of an amino acid with another amino acid that has the same net electronic charge and approximately the same size and shape. Amino acids with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Listed below are five groups of amino acids. Replacing an amino acid in a polypeptide or peptide with another amino acid from the same group results in a conservative substitution:

Group glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).

Group II: glutamic acid, aspartic acid and non-naturally occurring amino acids with carboxylic acid substituted C1-C4 aliphatic side chains (unbranched or one branch point).

Group III: lysine, ornithine, arginine and non-naturally occurring amino acids with amine or guanidino substituted C1-C4 aliphatic side chains (unbranched or one branch point).

Group IV: glutamine, asparagine and non-naturally occurring amino acids with amide substituted C1-C4 aliphatic side chains (unbranched or one branch point).

Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

A. Modified Thrombin Peptide Derivatives

In one embodiment of the invention, the thrombin peptide derivatives are modified relative to the thrombin peptide derivatives described above, wherein cysteine residues of thrombin peptide derivatives are replaced with amino acids having similar size and charge properties to minimize dimerization of the peptides. Examples of suitable amino acids include alanine, glycine, serine, or an S′-protected cysteine. Preferably, cysteine is replaced with alanine. The modified thrombin peptide derivatives have about the same biological activity as the unmodified thrombin peptide derivatives. See Publication No. US 2005/0158301 A1, which is hereby incorporated by reference.

It will be understood that the modified thrombin peptide derivatives disclosed herein can optionally comprise C-terminal amides and/or N-terminal acyl groups, as described above.

Preferably, the N-terminus of a thrombin peptide derivative is free (i.e., unsubstituted) and the C-terminus is free (i.e., unsubstituted) or amidated, preferably as a carboxamide (i.e., —C(O)NH₂).

In a specific embodiment, the modified thrombin peptide derivative comprises a polypeptide or peptide having the amino acid sequence of Arg-Gly-Asp-Ala-Xaa-X1-Gly-Asp-Ser-Gly-Gly-Pro-X2-Val (SEQ ID NO:10), or a C-terminal truncated fragment thereof having at least six amino acids. More specifically, the thrombin peptide derivative comprises the amino acid sequence of Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val (SEQ ID NO:11) or a fragment thereof comprising amino acids 10-18 of peptide. Even more specifically, the thrombin peptide derivative comprises the amino acid sequence Ala-Gly-Tyr-Lys-Pro-Asp-Glu-Gly-Lys-Arg-Gly-Asp-Ala-Xaa-X1-Gly-Asp-Ser-Gly-Gly-Pro-X2-Val (SEQ ID NO:12), or a fragment thereof comprising amino acids 10-18 of sequence. Xaa is alanine, glycine, serine or an S-protected cysteine. X1 is Glu or Gln and X2 is Phe, Met, Leu, His or Val. Preferably X1 is Glu, X2 is Phe, and Xaa is alanine. One example of a thrombin peptide derivative of this type is a polypeptide having the amino acid sequence Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val (SEQ ID NO:13). A further example of a thrombin peptide derivative of this type is the polypeptide H-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-NH₂ (SEQ ID NO:14). Another example of a thrombin peptide derivative of this type is the polypeptide H-Ala-Gly-Tyr-Lys-Pro-Asp-Glu-Gly-Lys-Arg-Gly-Asp-Ala-Ser-Glu-Gly-Asp-Ser-Gly-Gly-Pro-Phe-Val-NH₂ (SEQ ID NO:15). Zero, one, two or three amino acids in the thrombin peptide derivative differ from the amino acid at the corresponding position of the sequences, provided that Xaa is alanine, glycine, serine or an S-protected cysteine. Preferably, the difference is conservative as defined herein.

In another specific embodiment, the thrombin peptide derivative comprises a polypeptide having the amino acid sequence Asp-Asn-Met-Phe-Xbb-Ala-Gly-Tyr-Lys-Pro-Asp-Glu-Gly-Lys-Arg-Gly-Asp-Ala-Xaa-Glu-Gly-Asp-Ser-Gly-Gly-Pro-Phe-Val-Met-Lys-Ser-Pro-Phe (SEQ ID NO:16), or a fragment thereof comprising amino acids 6-28. More preferably, the thrombin peptide derivative comprises a polypeptide having the amino acid sequence Asp-Asn-Met-Phe-Xbb-Ala-Gly-Tyr-Lys-Pro-Asp-Glu-Gly-Lys-Arg-Gly-Asp-Ala-Xaa-X1-Gly-Asp-Ser-Gly-Gly-Pro-X2-Val-Met-Lys-Ser-Pro-Phe (SEQ ID NO:17), or a fragment thereof comprising amino acids 6-28, Xaa and Xbb are independently alanine, glycine, serine or an S-protected cysteine. X1 is Glu or Gln and X2 is Phe, Met, Leu, His or Val. Preferably X1 is Glu, X2 is Phe, and Xaa and Xbb are alanine. One example of a thrombin peptide derivative of this type is a polypeptide comprising the amino acid sequence Asp-Asn-Met-Phe-Ala-Ala-Gly-Tyr-Lys-Pro-Asp-Glu-Gly-Lys-Arg-Gly-Asp-Ala-AIa-Glu-Gly-Asp-Ser-Gly-Gly-Pro-Phe-Val-Met-Lys-Ser-Pro-Phe (SEQ ID NO:18). A further example of a thrombin peptide derivative of this type is the polypeptide COOH-Asp-Asn-Met-Phe-Ala-Ala-Gly-Tyr-Lys-Pro-Asp-Glu-Gly-Lys-Arg-Gly-Asp-Ala-Ala-Glu-Gly-Asp-Ser-Gly-Gly-Pro-Phe-Val-Met-Lys-Ser-Pro-Phe-NH₂ (SEQ ID NO:19). Zero, one, two or three amino acids in the thrombin peptide derivative can differ from the amino acid at the corresponding position of the sequences. Xaa and Xbb are independently alanine, glycine, serine or an S-protected cysteine. Preferably, the difference is conservative as in conservative substitutions of the thrombin peptide derivatives.

An “S-protected cysteine” is a cysteine residue in which the reactivity of the thiol moiety, —SH, is blocked with a protecting group. Suitable protecting groups are known in the art and are disclosed, for example, in Greene and Wuts, Protective Groups in Organic Synthesis, 3rd Edition, John Wiley & Sons, (1999), pp. 454-493. Suitable protecting groups should be nontoxic, stable in pharmaceutical formulations and have minimum additional functionality to maintain the activity of the thrombin peptide derivative. A free thiol can be protected as a thioether, a thioester, or can be oxidized to an unsymmetrical disulfide. Preferably the thiol is protected as a thioether. Suitable thioethers include, but are not limited to, S-alkyl thioethers (e.g., C1-C5 alkyl), and S-benzyl thioethers (e.g., cysteine-S—S-i-Bu). Preferably the protective group is an alkyl thioether. More preferably, the S-protected cysteine is an S-methyl cysteine. Alternatively, the protecting group can be: (1) a cysteine or a cysteine-containing peptide (the “protecting peptide”) attached to the cysteine thiol group of the thrombin peptide derivative by a disulfide bond; or (2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

B. Thrombin Peptide Derivative Dimers

In some aspects of the present invention, the thrombin peptide derivatives of the methods are thrombin peptide derivative dimers. See Publication No. US 2005/0153893, which is hereby incorporated by reference. The dimers essentially do not revert to monomers and still have about the same biological activity as the thrombin peptide derivatives monomer described above. A “thrombin peptide derivative dimer” is a molecule comprising two thrombin peptide derivatives linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention includes the thrombin peptide derivatives described above. Specifically, thrombin peptide derivatives have less than about fifty amino acids, preferably less than about thirty-three amino acids. Thrombin peptide derivatives also have sufficient homology to the fragment of human thrombin corresponding to thrombin amino acid residues 508-530: Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:1) so that the polypeptide activates NPAR.

In a specific embodiment, each thrombin peptide derivative comprising a dimer comprises a polypeptide having the amino acid sequence Arg-Gly-Asp-Ala-Cys-X1-Gly-Asp-Ser-Gly-Gly-Pro-X2-Val (SEQ ID NO:4), or a C-terminal truncated fragment thereof comprising at least six amino acids. More specifically, each thrombin peptide derivative comprises the amino acid sequence of Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:5), or a fragment thereof comprising amino acids 10-18. Even more specifically, the thrombin peptide derivative comprises the amino acid sequence Ala-Gly-Tyr-Lys-Pro-Asp-Glu-Gly-Lys-Arg-Gly-Asp-Ala-Cys-X1-Gly-Asp-Ser-Gly-Gly-Pro-X2-Val (SEQ ID NO:8), or a fragment thereof comprising amino acids 10-18. X1 is Glu or Gln and X2 is Phe, Met, Leu, His or Val. Preferably X1 is Glu, and X2 is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val (SEQ ID NO:1). A further example of a thrombin peptide derivative of this type is a polypeptide having the amino acid sequence H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-NH₂ (SEQ ID NO:1; TP508). Zero, one, two or three amino acids in the thrombin peptide derivative differ from the amino acid at the corresponding position of the sequences. Preferably, the difference is conservative as for conservative substitutions of the thrombin peptide derivatives.

In another specific embodiment, each thrombin peptide derivative comprising a dimer comprises a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:20), or a C-terminal truncated fragment thereof having at least twenty-three amino acids. More preferably, each thrombin peptide derivative comprises the amino acid sequence Ala-Gly-Tyr-Lys-Pro-Asp-Glu-Gly-Lys-Arg-Gly-Asp-Ala-Cys-X1-Gly-Asp-Ser-Gly-Gly-Pro-X2-Val-Met-Lys-Ser-Pro-Phe-Asn-Asn-Arg-Trp-Tyr (SEQ ID NO:21), or a C-terminal truncated fragment thereof comprising at least twenty-three amino acids. X1 is Glu or Gln and X2 is Phe, Met, Leu, His or Val. Preferably X1 is Glu, and X2 is Phe. One example of a thrombin peptide derivative of this type is a polypeptide comprising the amino acid sequence Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr (SEQ ID NO:22). A further example of a thrombin peptide derivative of this type is a polypeptide comprising the amino acid sequence H-Ala-Gly-Tyr-Lys-Pro-Asp-Glu-Gly-Lys-Arg-Gly-Asp-Ala-Cys-Glu-Gly-Asp-Ser-Gly-Gly-Pro-Phe-Val-Met-Lys-Ser-Pro-Phe-Asn-Asn-Arg-Trp-Tyr-NH₂ (SEQ ID NO:23). Zero, one, two or three amino acids in the thrombin peptide derivative differ from the amino acid at the corresponding position of the sequences. Preferably, the difference is conservative as defined for conservative substitutions of the thrombin peptide derivatives.

An “effective amount” is the quantity of the thrombin peptide derivative described herein that results in an improved clinical outcome of the condition being treated with the thrombin peptide derivative compared with the absence of treatment. The amount of the thrombin peptide derivative administered will depend on the degree, severity, and type of the disease or condition, the amount of therapy desired, and the release characteristics of the pharmaceutical formulation. It will also depend on the subject's health, size, weight, age, sex and tolerance to drugs.

Typically, the thrombin peptide derivative is administered for a sufficient period of time to achieve the desired therapeutic effect. Typically, from about 1 μg per day to about 1 mg per day of the thrombin peptide derivatives (preferably from about 5 μg per day to about 100 μg per day) is administered to the subject in need of treatment, especially for a local means of administration. The thrombin peptide derivatives can also be administered at a dose of from about 0.1 mg/kg/day to about 15 mg/kg/day, with from about 0.2 mg/kg/day to about 3 mg/kg/day being preferred, especially for systemic means of administration. Typical dosages for the thrombin peptide derivative of the invention are also 5-500 mg/day, preferably 25-250 mg/day, especially for systemic means of administration.

“Treating” means that following a period of administering the thrombin peptide derivative or composition comprising a thrombin peptide derivative, a beneficial therapeutic and/or prophylactic result is achieved, which can include a decrease in the severity of symptoms or delay in or inhibition of the onset of symptoms, increased longevity and/or more rapid or more complete resolution of the disease or condition, or other improved clinical outcome as measured according to the site that is being observed or the parameters measured for a particular disease or disorder. “Reducing the risk” refers to decreasing the probability of developing a disease, disorder or medical condition, in a subject, wherein the subject is, for example, a subject who is at risk for developing the disease, disorder or condition.

The disclosed thrombin peptide derivative can be administered by any suitable route, locally (e.g., topically) or systemically, including, for example, by parenteral administration.

Parenteral administration can include, for example, intramuscular, intravenous, subcutaneous, or intraperitoneal injection or vascular administration, and can also include transdermal patch and implanted slow-release devices such as pumps. Topical administration can include, for example, creams, gels, ointments or aerosols. Respiratory administration can include, for example, inhalation or intranasal drops. For certain indications, it is advantageous to inject or implant the thrombin peptide derivative directly to the treatment site. The thrombin peptide derivative can be advantageously administered in a sustained release formulation. The thrombin peptide derivative can be administered chronically, wherein the peptide derivative is administered over a long period of time (at least 60 days, but more typically, for at least one year), at intervals or by a 660 continuous delivery method, to treat a chronic or recurring disease or condition.

The thrombin peptide derivative can be administered to the subject in conjunction with an acceptable pharmaceutical carrier as part of a pharmaceutical composition. The formulation of the pharmaceutical composition will vary according to the route of administration selected. Suitable pharmaceutical carriers may contain inert ingredients which do not interact with the compound. The carriers should be biocompatible, i.e., non-toxic, non-inflammatory, non-immunogenic and devoid of other undesired reactions at the administration site. Examples of pharmaceutically acceptable carriers include, for example, saline, aerosols, commercially available inert gels, or liquids supplemented with albumin, methyl cellulose or a collagen matrix. Standard pharmaceutical formulation techniques can be employed, such as those described in Remington's Pharmaceutical Sciences, Mack Publishing Company, Easton, Pa. Other suitable pharmaceutical carriers include those described in U.S. Pat. No. 7,294,596, the entire teaching of which is incorporated herein by reference.

The compositions used in the methods of the present invention can additionally comprise a pharmaceutical carrier in which the thrombin peptide derivative is dissolved or suspended. Examples of pharmaceutically acceptable carriers include, for example, saline, aerosols, commercially available inert gels, or liquids supplemented with albumin, methyl cellulose or a collagen matrix. Typical of such formulations are gels. Gels are comprised of a base selected from an oleaginous base, water, or an emulsion-suspension base, as previously described. To the base is added a gelling agent that forms a matrix in the base, increasing its viscosity to a semisolid consistency. Examples of gelling agents are hydroxypropyl cellulose, acrylic acid polymers, and the like. The active ingredients are added to the formulation at the desired concentration at a point preceding addition of the gelling agent or can be mixed after the gelation process.

Injectable delivery formulations may be administered intravenously or directly at the site in need of treatment. The injectable carrier may be a viscous solution or gel.

Delivery formulations include physiological saline, bacteriostatic saline (saline containing about 0.9% mg/mL benzyl alcohol), phosphate-buffered saline, Hank's solution, Ringer's-lactate, or liquids supplemented with albumin, methyl cellulose, or hyaluronic acid. Injectable matrices include polymers of poly(ethylene oxide) and copolymers of ethylene and 690 propylene oxide (see Cao et al, J. Biomater. Sci 9:475 (1998) and Sims et al, Plast Reconstr. Surg. 98:843 (1996), the entire teachings of which are incorporated herein by reference).

Methods for encapsulating compositions (such as in a coating of hard gelatin or cyclodextran) are known in the art (Baker, et al, “Controlled Release of Biological Active Agents”, John Wiley and Sons, 1986).

Ointments are typically prepared using an oleaginous base, e.g., containing fixed oils or hydrocarbons, such as white petrolatum or mineral oil, or an absorbent base, e.g., consisting of an absorbent anhydrous substance or substances, for example anhydrous lanolin. Following formation of the base, the active ingredients are added in the desired concentration.

Creams generally comprise an oil phase (internal phase) containing typically fixed oils, 700 hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate; hydrophilic colloids, such as acacia colloidal clays, beegum, and the like. Upon formation of the emulsion, the active ingredients are added in the desired concentration.

Gels contain a base selected from an oleaginous base, water, or an emulsion-suspension base, as previously described. To the base is added a gelling agent which forms a matrix in the base, increasing its viscosity to a semisolid consistency. Examples of gelling agents are hydroxypropyl cellulose, acrylic acid polymers, and the like. The active ingredients are added to 710 the formulation at the desired concentration at a point preceding addition of the gelling agent.

A thrombin peptide derivative can be administered to a subject alone or in combination with one or more other therapeutics, for example, a cholesterol-lowering agent, an anti-hypertensive agent, a beta-blocker, an anti-coagulant, a thrombolytic agent, an analgesic, an anti-inflammatory agent, an anti-plaque agent, insulin, a nitric oxide generating agent, an antiviral agent or an antibiotic. In one method, a thrombin peptide derivative can be administered to a subject in combination with an antiviral that is effective against coronavirus.

Thrombin peptide derivatives and modified thrombin peptide derivatives can be synthesized by solid phase peptide synthesis (e.g., BOC or FMOC) method, by solution phase synthesis, or by other suitable techniques including combinations of the foregoing methods. The BOC and FMOC methods, which are established and widely used, are described in Merrifield, J. Am. Chem. Sot: 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al, Synthesis, J: 315 (1992)). The teachings of these six articles are incorporated herein by reference in their entirety.

Thrombin peptide derivative dimers can be prepared by oxidation of the monomer. Thrombin peptide derivative dimers can be prepared by reacting the thrombin peptide derivative with an excess of oxidizing agent. A well-known suitable oxidizing agent is iodine.

A “non-aromatic heterocyclic group” as used herein, is a non-aromatic carbocyclic ring system that has 3 to 10 atoms and includes at least one heteroatom, such as nitrogen, oxygen, or sulfur. Examples of non-aromatic heterocyclic groups include piperazinyl, piperidinyl, pyrrolidinyl, morpholinyl, thiomorpholinyl.

An “alkyl” is a straight chain or branched saturated hydrocarbon radical. Typically, an alkyl group has from 1 to about 10 carbon atoms, preferably from 1 to about 4 carbon atoms. Exemplary alkyl groups include, but are not limited to, methyl, ethyl, n-propyl, iso-propyl, n-butyl, sec-butyl, tert-butyl, pentyl, cyclopentyl, hexyl, cyclohexyl, octyl and cyclooctyl.

### II. Coronavirus

A non-limiting example of an etiologic agent resulting in an ARDS pathology is a Coronavirus, Coronaviruses (order Nidovirales, family Coronaviridae) are a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996, Coronaviridae: the viruses and their replication, p. 1075-1094, Fields Virology, Lippincott-Raven, Philadelphia, Pa.). However, the natural host range of each coronavirus strain is narrow, typically consisting of a single species. Coronaviruses typically bind to target cells through Spike-receptor interactions and enter cells by receptor mediated endocytosis or fusion with the plasma membrane (Holmes, et al., 1996, supra).

Upon entry into susceptible cells, the open reading frame (ORF) nearest the 5′ terminus of the coronavirus genome is translated into a large polyprotein. This polyprotein is autocatalytically cleaved by viral-encoded proteases, to yield multiple proteins that together serve as a virus-specific, RNA-dependent RNA polymerase (RdRP). The RdRP replicates the viral genome and generates 3′ coterminal nested subgenomic RNAs. Subgenomic RNAs include capped, polyadenylated RNAs that serve as mRNAs, and antisense subgenomic RNAs complementary to mRNAs. In one embodiment, each of the subgenomic RNA molecules shares the same short leader sequence fused to the body of each gene at conserved sequence elements known as intergenic sequences (IGS), transcriptional regulating sequences (TRS) or transcription activation sequences. It has been controversial as to whether the nested subgenomic RNAs are generated during positive or negative strand synthesis; however, recent work favors the model of discontinuous transcription during minus strand synthesis (Sawicki, et al., 1995, Adv. Exp. Med. Biol. 380:499-506; Sawicki and Sawicki Adv. Expt. Biol. 1998, 440:215).

A SARS-CoV-2 reference sequence can be found in GenBank accession NC_045512.2 as of Mar. 2, 2020. This sequence is a 29903 bp ss-RNA and is referred to as the Wuhan seafood market pneumonia virus isolate Wuhan-Hu-1. The virus is Severe acute respiratory syndrome coronavirus 2 (SARS-CoV-2) with the taxonomy of Viruses; Riboviria; Nidovirales; Cornidovirineae; Coronaviridae; Orthocoronavirinae; Betacoronavirus; Sarbecovirus. (Wu et al. “A novel coronavirus associated with a respiratory disease in Wuhan of Hubei province, China” Unpublished; NCBI Genome Project, Direct Submission, Submitted (17 Jan. 2020) National Center for Biotechnology Information, NIH, Bethesda, MID 20894, USA; Wu et al. Direct Submission, Submitted (5 Jan. 2020) Shanghai Public Health Clinical Center and School of Public Health, Fudan University, Shanghai, China).

The genome of SARS-CoV-2, with reference to GenBank accession NC_045512.2 as of Mar. 2, 2020, includes (1) a 5′UTR 1-265), (2) Orflab gene (266-21555), S gene encoding a spike protein (21563..25384), ORF3a gene (25393..26220), E gene encoding E protein (26245..26472), M gene (26523..27191), ORF6 gene (27202..27387), ORF7a gene (27394..27759), ORF7b gene (27756..27887), ORF8 gene (27894..28259), N gene (28274..29533), ORF10 gene (29558..29674), and 3′UTR (29675..29903).

The term “coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2) N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (GenBank accession number NC_045512.2), CoV (ATCC accession #VR-1475), human CoV 229E (ATCC accession #VR-740), human CoV OC43 (ATCC accession #VR-920), and SARS-coronavirus (Center for Disease Control).

### III. Acute Respiratory Distress Syndrome (ARDS)

Acute respiratory distress syndrome (“ARDS”) is a manifestation of a systemic inflammatory response that develops, for example, as a consequence of direct or indirect lung injury e.g., in both medical and surgical patients. The hallmark of ARDS is deterioration in blood oxygenation and respiratory system compliance as a consequence of permeability edema.

A consensus definition of ARDS, as recommended in 1994 by the American-European Consensus Conference Committee, distinguishes ARDS from other conditions such as acute lung injury (ALI) based on differing severity of clinical lung injury: patients with less severe hypoxemia are considered to have ALI, and those with more severe hypoxemia are considered to have the ARDS. As a consequence, ARDS is defined by the following criteria (Bernard et al., Am. J. Respir. Crit. Care Med 149, 818-824, 1994): 1. Acute onset; 2. Bilateral infiltrates on chest radiography; 3. Pulmonary-artery wedge pressure is less than or equal to 18 rum Hg or the absence of clinical evidence of left atrial hypertension; and 4. hypoxemia, as determined by the ratio of partial pressure of arterial oxygen to fraction of inspired oxygen, i.e., PaO2:FiO2, is less than or equal to 200. ARDS is often progressive, characterized by distinct stages exhibiting different clinical, histopathological and radiographic parameters. An acute phase of ARDS involves acute neutrophil influx to the lungs e.g., arising from e.g., sepsis, pneumonia, aspiration, ischemia (circulatory arrest, hemorrhagic shock), trauma, severe asthma, poisoning, severe acute respiratory syndrome (SARS), influenza, or infection.

The acute phase of ARDS is characterized by rapid onset of respiratory failure in a patient having a predisposition for the condition, especially arterial hypoxemia that is refractory to oxygen supplementation. Broncho-alveolar-lavage (BAL) studies reveal substantial inflammation in areas that appear normal by radiography or tomography as well as in areas that exhibit alveolar filling, consolidation, and atelectasis. Pathologically, the lung in this acute phase exhibits diffuse alveolar damage, with neutrophils, macrophages, erythrocytes, hyaline membranes, capillary injury, and disruption of the alveolar epithelium.

More particularly, the acute phase of the condition is characterized by sloughing of the bronchiolar and alveolar epithelial cells, with the formation of protein-rich hyaline membranes on the basement membrane. Neutrophils have been detected adhering to the injured capillary endothelium and marginating through the interstitium into the air space, which is filled with edema fluid. In the air space, alveolar macrophages secrete cytokines such as the interleukins IL-1, IL-6, IL-8 and IL-10, and tumor necrosis factor-α (TNF-α), which act locally to stimulate chemotaxis and activate neutrophils to release oxidants, proteases, leukotrienes, and other pro-inflammatory molecules such as platelet activating factor (PAF). The production of proinflammatory cytokines, and the balance between proinflammatory cytokines and anti-inflammatory mediators e.g., IL-1 receptor antagonist, soluble TNF, autoantibodies against IL-8, and anti-inflammatory cytokines IL-10 and IL-11 determine the extent of inflammatory response. The inflammatory response may result in vascular leakage of plasma proteins into the alveolar spaces of the lungs causing lung edema.

The acute phase may progress to fibrosing alveolitis with persistent hypoxemia, increased alveolar dead space and further decrease in alveolar compliance. In patients with ARDS the microvascular, interstitial, and alveolar spaces of the lungs are the primary targets for fibrin deposition, and micro thrombus formation can occur in multiple organs, with lungs and kidneys as the most exposed, leading to multiple organ failure (MOF). Pulmonary hypertension may arise from obliteration of the pulmonary capillary bed and, in severe cases this may cause right ventricular failure. Pneumothorax may occur in about 10-13% of subjects.

In subjects who recover, there is gradual resolution of hypoxemia and improved lung compliance and pulmonary function may be restored to normal in some subjects. In most subjects who survive ARDS, pulmonary function can take 6-12 months to be restored to nearly normal levels. Residual impairment of pulmonary mechanics may include mild restriction, obstruction, impairment of the diffusing capacity for carbon monoxide, or gas-exchange abnormalities with exercise, but these abnormalities are usually asymptomatic. Severe disease and prolonged mechanical ventilation identify patients at highest risk for persistent abnormalities of pulmonary function. Those who survive the illness have a reduced health-related quality of life as well as pulmonary-disease-specific health-related quality of life.

Certain embodiments are directed to methods of treatment of ARDS, particularly virus induced ARDS, and/or one or more complications thereof or for the prophylactic treatment of one or more clinical disorders associated with the development of ARDS. In certain aspects, the method comprising administering to a subject in need thereof a formulation comprising TP508 or derivative thereof for a time and under conditions sufficient to reduce or prevent ARDS related pathology.

There is often a delay between a precipitating factor e.g., trauma, poisoning, viral infection, etc. and the onset of ARDS, which the inventors reason provides a window of opportunity for administering a formulation of the invention or other formulation comprising TP508 or a derivative thereof. Accordingly, in one example, this invention provides a method for the prophylaxis or prevention of ARDS comprising administering to a subject at risk of developing ARDS or exposed to one or more risk factors of ARDS (e.g., infection by a respiratory virus such as SARS-CoV-2) a formulation comprising TP508 or derivative thereof. In certain aspects, the subject is suffering from breathing difficulty and/or has reduced breathing capability. In certain aspects, the subject can inhale the formulation and the formulation is administered to the subject by inhalation. In another example, the formulation is administered by injection. The formulation can be administered to the subject by injection via an intravenous, intraperitoneal, intramuscular, or subcutaneous route.

As used herein the term “treatment” includes therapeutic treatment of a subject who has already suffered ARDS or a complication thereof including neutrophilic inflammation and its downstream consequences such as, for example, alveolar filling, alveolar epithelial damage or loss, amongst others, and prophylactic treatment of a subject having one or more risk factors for ARDS but that has not yet suffered an acute phase of ARDS or a complication thereof. In this respect, it will be evident that the reduction of neutrophilic inflammation and enhancement/induction of alveolar re-epithelialization are more pertinent to therapeutic regimens, and that the prevention of neutrophilic inflammation and/or the prevention or reduction of alveolar epithelial injury or loss are more pertinent to prophylactic regimens. Consistent with this construction, the term “prevent” or “prevention” as used throughout this specification shall not be taken to require an absolute i.e., 100% abrogation of neutrophilic inflammation or epithelial damage/loss in a subject, and it is sufficient that there is a significant reduction in these adverse consequences of ARDS using the method and formulations of the present invention compared to the absence of treatment in accordance with the present invention. Similarly, the term “reduction” or “reduce” as used throughout this specification shall not be taken to require an abrogation of neutrophilic inflammation or epithelial damage/loss in a subject more than a significant effect compared to the absence of treatment in accordance with the present invention. Similarly, the terms “enhance”, “enhancement”, “induce” and “induction” as used throughout this specification shall not be taken to require any particular quantitative change, merely an improvement that is significant compared to the absence of treatment in accordance with the present invention. The term “enhance” and “enhancement” will be understood or taken to mean an increase in the level or amount of a stated integer that is already present whereas the terms “induce” and “induction” refer to the increase in level or amount of an integer that is not detectable prior to the induction, however may be present in undetectable amounts.

As used herein, the term “administer” shall be taken to mean that a formulation is applied to the respiratory system of a subject including the nasal passage, buccal cavity, throat or esophagus or lung, by inhalation and/or applied to the circulatory system of a subject by injection intramuscularly, subcutaneously, intravenously, intraperitoneally etc., including single or repeated or multiple dosages by any administration route. As used herein the term “inhalation” shall be taken to include “aspiration”.

As used herein, the term “subject in need thereof” shall be taken to mean a subject that has developed or suffers from ARDS or one or more complications thereof or is predisposed by virtue of having one or more risk factors to suffering from ARDS or one or more complications thereof. In one example, a subject has not yet suffered significant impairment of breathing or significant damage to the alveolar epithelium and has one or more risk factors for ARDS or acute lung injury or a complication thereof, such as diagnosis of a respiratory virus infection.

The present invention clearly contemplates repeated administration of a formulation as described herein according to any embodiment in the therapy or prophylaxis of ARDS and complications thereof. For example, repeated injection and/or inhalation of a formulation of the present invention may be required to reduce or prevent inflammatory responses in the lung for a long period of time, e.g., during sepsis or persistent or long-term infection by a bacterial agent or virus.

Repeated administration of a formulation as described herein may be timed to ensure a sufficiently high concentration of the bioactive peptide component of the formulation in plasma of the subject and/or at the site of action in the treatment regimen. For example, second and/or subsequent doses of a peptide formulation of the invention as described according to any embodiment hereof may be administered at a time when serum concentration of a peptide provided by one or more previous doses fall(s) below a desired level at which it is active or provides sufficient benefit to the patient. Such booster doses of a peptide formulation of the present invention are clearly contemplated in the prophylaxis and/or therapy of ARDS and/or one or more complications thereof according to the present invention.

For example, the present invention provides a method of treatment or prophylaxis of a subject in need thereof, said method comprising:

(i) identifying a subject suffering from ARDS and/or one or more complications thereof or one or more clinical disorders associated with the development of ARDS or is at risk of suffering from ARDS and/or one or more complications thereof or one or more clinical disorders associated with the development of ARDS; and (ii) administering a formulation as described herein to the subject.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, considering the present disclosure, appreciate that many changes can be made in the specific embodiments which are disclosed and still obtain a like or similar result without departing from the spirit and scope of the invention.

### Example 1

The TP508 peptide is GMP manufactured and available for testing TP508 has completed human clinical trials for diabetic foot ulcers (IND 56,811, Phase 2) and distal radius fractures (IND 59,066, Phase 2 and Phase 3), and non-clinical safety/toxicology and PK studies with no reported drug-related adverse events. Thus, this is a drug that can be rapidly redirected into clinical trials for COVID-19 to impact the current and any future viral pandemics. The studies outlined below suggest that TP508 has potential to mitigate lethal effects of SARS-CoV-2-induced ARDS and to help restore normal pulmonary function in those that survive.

A. TP508 Inhibits TNFα Effects on Pulmonary Endothelial Cell Functions

SARS-CoV-2 causes an exaggerated proinflammatory cytokine response with increased TNFα, IFN-γ, IL-10, and IL-6 that damage alveolar endothelial and epithelial cells. Endothelial cell damage causes vascular fluid leakage that fills the alveoli and promotes neutrophil and leukocyte infiltration. As fluid and cells enter the alveoli, apoptosis of alveolar epithelial cells occurs causing further alveolar breakdown, failure of oxygen exchange, and systemic hypoxia leading to mortality. Since TNFα appears to be a major contributor to ARDS progression, the effects of TP508 on TNFα induced vascular changes was evaluated.

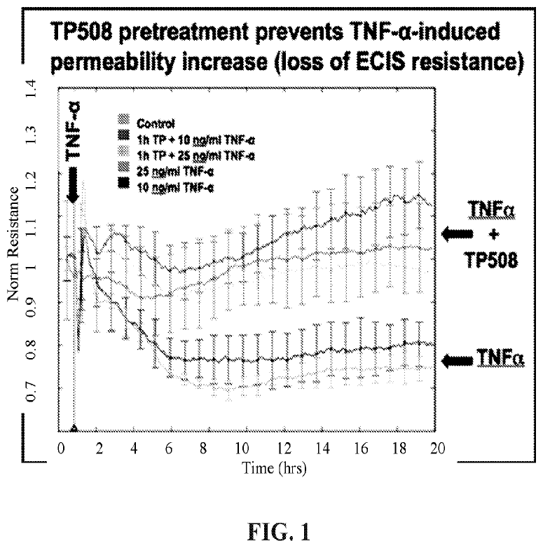

TP508 Effects on Vascular Permeability. Treating human pulmonary endothelial cells with TP508 prevented TNFα-induced changes in vascular permeability (FIG. 1). In this analysis, vascular permeability was measured by culturing a monolayer of human pulmonary endothelial cells and then measuring the electrical resistance of the monolayer. As shown, incubation with TNFα causes a decrease in the electrical resistance as gaps are induced between the endothelial cells. Pretreating the cultures with TP508 prevents the gap-forming effects of TNFα thus maintaining the cell-cell junctions, barrier function, and electrical resistance.

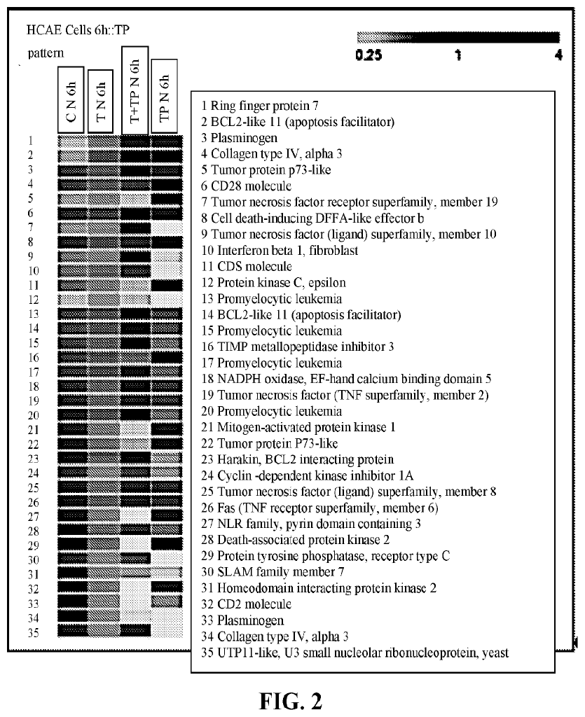

Gene Expression in Endothelial Cells. Gene array analysis identified a family of genes that are upregulated in human coronary artery endothelial cells (HCAE cells) by TNFα within 6 hours of treatment, but not upregulated by TNFα when cells are pre-treated with TP508. FIG. 2 shows a heat map of 35 genes that were upregulated by TNFα (TN 6 h) by 2 to 10-fold over control cells (CN 6 h). As shown, TP508 pretreatment prevents the TNFα-induced upregulation (T+TP N 6 h) and does not by itself (TP N 6 h) upregulate the molecules. Molecules in this group include adhesion molecules, pro-inflammatory signaling molecules, and pro-apoptotic molecules induced by TNFα. Thus, TP508 reverses many of the damaging effects of TNFα on endothelial cells. Because of the role of TNFα in ARDS progression, it is believed that TP508 will also prevent progression of disease in COVID-19 patients.

One of the specific genes shown in FIG. 2 that is upregulated by TNFα but inhibited to levels at or below controls by TP508 is the alpha 3 subunit of Collagen IV which is known as the Goodpasture's antigen (Genes 34 in FIG. 2). Goodpasture syndrome is an autoimmune disease known as the anti-glomerular basement membrane disease in which antibodies attack the basement membrane in lungs and kidneys leading to diffuse alveolar hemorrhage, glomerulonephritis and congestive heart failure. The damage from exposure of this subunit of collagen and antibody binding can quickly result in permanent damage to lung, kidney and other organs often leading to death. Interestingly, the lesions caused by Goodpasture syndrome were also described by Goodpasture as being a common feature of those dying from influenza-induced pneumonia in 1919. His conclusion was that these lesions were the result of viral-induced inflammation. Microscopically the lungs showed an extreme degree of injury and destruction of alveolar walls with hemorrhage, edema, a little fibrin and cellular exudate. The alveolar ducts were dilated and on the walls of some of them was found the typical hyaline membrane of that seen in Goodpasture Syndrome.

Goodpasture also reported similar lesions in the kidney, suggesting that either the virus spread, or that the systemic inflammatory response contributed to similar lesions in other organs. Thus, the systemic upregulation of this gene by TNFα may contribute to both pulmonary and multiple organ failure. That TP508 blocks this upregulation in endothelial cells in HCAE cells is another indication that TP508 may help prevent progressive loss of pulmonary function and multiple organ failure caused by systemic effects of TNFα or other inflammatory cytokines in response to viral infection.

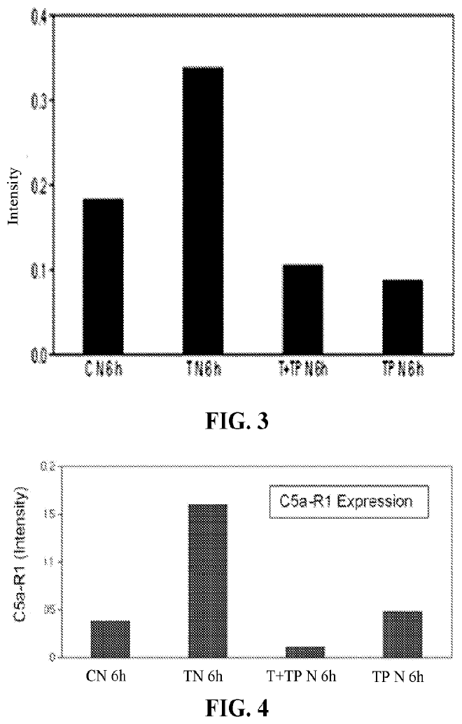

Gene array data also demonstrated that TP508 inhibited TNFα-induced upregulation of TGFβ. In the lung, TGFβ suppresses proliferation of epithelial type II (surfactant producing) cells and promotes epithelial-mesenchymal transition, fibroblast activation, and the reorganization of extracellular matrix. These effects of TGFβ contribute to lung tissue remodeling and chronic pulmonary fibrosis and emphysema. As shown in FIG. 3, when human endothelial cells were treated with TP508 prior to TNFα treatment, the upregulation of TGFβ2 measured 6 hours later was prevented. In fact, TP508 appeared to suppress TGFβ2 expression below that seen in controls. These in vitro results suggest that treating patients with ARDS or SARS-CoV-2 infections with TP508 may help prevent pulmonary fibrosis and other delayed effects that reduce lung function in patients that survive the disease.

Gene array data also demonstrated that TP508 inhibited TNFα-induced upregulation of complement 5a receptor 1 (C5a-R1) which is involved in activating the cytokine storm following SARS-CoV-2 C5a leads to exaggerated proinflammatory responses and neutrophil and macrophage activation with release of histones and reactive oxygen species that lead to endothelial damage inflammation and thrombosis. As shown in FIG. 4, when human endothelial cells were treated with TP508 prior to TNFα treatment, the upregulation of C5a-R1 measured 6 hours later was prevented. These results suggest that treating COVID-19 patients with TP508 may reduce the SARS-CoV-2-induced cytokine storm and help prevent coagulation by preventing complement changes in the endothelial cells.

B. TP508 Reverses Endothelial Dysfunction In Vivo

TP508 Restores NO Production and Overcomes Effects of Comorbidities. SARS-CoV-2-induced pulmonary failure is much higher in elderly patients with preexisting hypertension, diabetes, cardiovascular or chronic pulmonary disease. All of these comorbidities are associated with endothelial dysfunction and loss of NO signaling. TP508 stimulates NO production and restores NO signaling to restore endothelial function and reverse effects of these comorbidities. Experiments with isolated vascular segments demonstrated that TP508 treatment increased NO-dependent vascular smooth muscle relaxation, thus having potential to decrease hypertension and airway constriction. TP508 injection directly into heart tissue with chronic ischemia restored myocardial function and restored vascular nitric oxide (NO) signaling. Additionally, in normal, diabetic, and hypercholesteremic minipigs, intravenous (IV) injection of TP508 reduced the effects of acute myocardial infarct, restored vascular function, and increased NO signaling. Since diabetes and high cholesterol-induced vascular dysfunction increase the risk of mortality from SARS-CoV-2 infection, these studies are important predictors of TP508 efficacy in restoring NO signaling and reducing progression of disease in “at risk” populations.

TP508 Stimulation of NO Production Could Decrease SARS-CoV-2 Replication and Infectivity. Three clinical trials were initiated recently to determine if inhaled NO would decrease COVID-19 progression toward pulmonary failure and death. The basis for these trials is that NO increases vascular perfusion, prevents airway constriction, and may have direct antiviral activity. A pilot clinical study conducted in patients with the original SARS virus indicated that NO gas inhalation reduced patient ventilator time and suggested a positive effect on pulmonary outcome. Additional in vitro studies showed that NO donor molecules or the activation of nitric oxide synthase inhibited the viral replication cycle of SARS CoV-1 and reduced viral infectivity. Since SARS CoV-1 and SARS-CoV2 share much of their genetic makeup and produce similar pathologies in the lung, it is likely that stimulation of NO production itself may reduce the infective damage of SARS-CoV-2. Thus, TP508 stimulation of NO production in endothelial cells may have a protective effect in the lungs of COVID-19 patients and could be used in combination with therapies including antivirals.

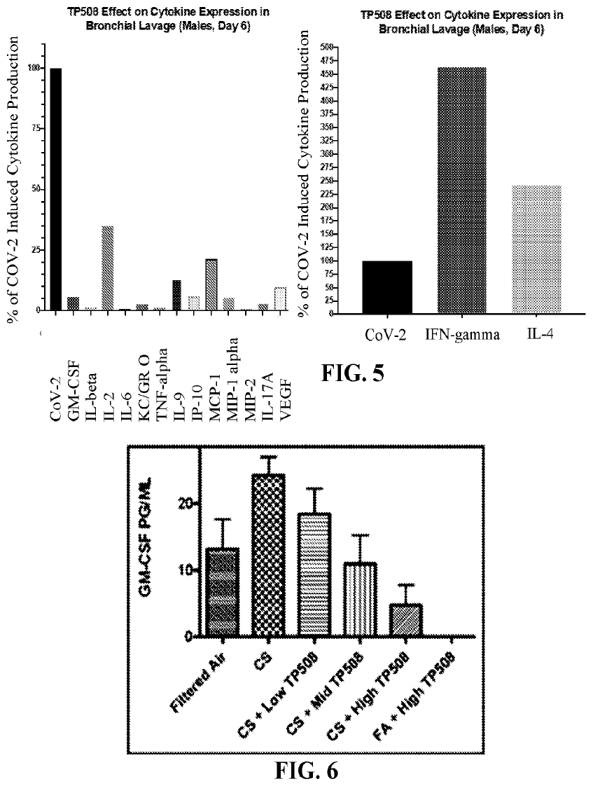

TP508 Reduces SARS-CoV2 Lung Inflammation by Inhibiting the “Cytokine Storm” induced by COVID-19 in ACE2 Transgenic Mice. A study was conducted at study at Lovelace Biomedical using the K018-hACE2 Transgenic (B6.Cg-Tg (Ki1-ACE2)2Prlmn/J) mice. In this model, expressing human ACE2 is expressed in mouse lung epithelial cells, SARS-CoV-2 infection causes animal death within 6 to 7 days and a large inflammatory response similar to that see in human COVID-19 patients. Mice were euthanized on Day-6 to evaluate effects on lung inflammation and cytokine expression. Injection of TP508 (SC) 24 hours after SARS-CoV-2 infection reduced the CoV-2-induced loss of body temperature and increased survival of male mice (˜25%, ns) through day 6 relative to viral challenged mice injected with saline (not shown). In these mice, TP508 reduced CoV-2-induced upregulation of major pro-inflammatory cytokines in BALF at day 6 by 70 to 99% relative to that seen in the viral challenged animals. Among these, IL-1β, IL-6, TNF-α, MIP1/2, and IL-17A (major contributors to cytokine effects of COVID-19 in humans) were all reduced by over 95% (FIG. 5). In contrast, the levels of anti-inflammatory and protective cytokines including IFN-γ and IL-4, were increased from 2.5 to 4-fold by TP508 treatment. These studies indicate that TP508 treatment prevents the viral induced “cytokine storm” and helps prevent COVID-19 progression.

C. TP508 Stimulation of Stem Cells to Regenerate Lung Tissue

The lung is a complex organ that utilizes self-renewing intrapulmonary epithelial stem cell populations, endothelial progenitor cells, and stem cells recruited from bone marrow to maintain function and recover from injury. Type II pneumocytes and bronchoalveolar stem cells (BASCs) are pulmonary progenitor cells that are critical in maintenance and restoration of pulmonary function. Studies showed that the original SARS-CoV targeted these cells early in the infection process and modified miRNA differentiation control pathways to prevent cell activation, therefore preventing normal anti-viral and tissue regenerative effects. The inventors anticipate that the alveolar destruction seen with SARS-CoV-2 is also associated with this targeting of endogenous pulmonary stem cells. Therefore, it is crucial that drugs are developed that can protect these cells and restore pulmonary function.

Extensive research in the therapeutic use of stem cells injected into the blood stream in ARDS animal studies has shown promise. Yet, the stem cell approach to treatment of ARDS and SARS in patients remains to be established. A few SARS-CoV-2 infected patients in China were treated with stem cells with apparent positive results, providing a basis for initiation of COVID-19 clinical trials with injection of isolated stem cells or progenitor cells. Even if these trials are successful, however, it is not clear whether there could possibly be enough cells to thwart effects of a pandemic such as COVID-19.

TP508 stimulates proliferation and activation of stem cells where they exist in the body to accelerate tissue regeneration following injury, ischemia, or radiation exposure. Examples of where TP508 injection has activated stem cells to regenerate tissues include: (1) diabetic mice and mice treated with whole body radiation where TP508 injection activated bone marrow stem cell proliferation and mobilization to accelerate healing; (2) models of whole brain radiation therapy where TP508 injection at the time irradiation stimulated proliferation of stem cells in the hippocampus of mice, decreased neural inflammation, and restored neural function; and (3) nuclear countermeasure studies where TP508 injection 24 hours after lethal doses of whole body radiation stimulated proliferation and differentiation of colon and intestinal stem cells to maintain GI barrier function and significantly increase survival. Thus, TP508 given systemically acts at sites of tissue injury to stimulate restorative responses that include activation of resident stem cells and recruitment of stem cells from bone marrow to restore function. These results suggest that TP508 injection during SARS-CoV-2 infections will help restore normal pulmonary repair processes and prevent loss of pulmonary function.

D. TP508 as a Medicinal Countermeasure for Acute Radiation Syndrome (ARS) and ARDS

SARS-CoV-2 infection causes severe acute respiratory stress (SARS) in the lower lung and in many cases progresses to ARDS, multiple organ failure, and death. This ARDS response and multiple organ failure caused by SARS-CoV-2 is similar to terminal effects of ARS. In both cases, there is an exaggerated inflammatory and pro-thrombotic reaction that elicits disruption of the alveolar-capillary membrane and vascular fluid leak. Dysfunction of endothelial cells plays a central role in the pathogenesis of ARDS. As described below, animal models of radiation injury demonstrate that a single injection of TP508 restores the integrity of the endothelial barrier and prevents ARS-induced mortality.

TP508 Prevents Radiation Combined Injury (RCI) Mortality. It is well established that ARS combined with dermal injury (RCI) is more lethal than ARS alone due to the enhanced cytokine response caused by the combination of radiation damage and wounding. Studies showed that TP508 applied topically to wounds or injected systemically restored normal healing and significantly increased survival. The increase in survival was at least in part due to TP508 preventing the RCI-induced increase in IL-6 and other cytokines. Thus, TP508 reduced the aberrant cytokine storm caused by RCI, suggesting that it could have a similar immune modulating effect on patients infected with SARS-CoV-2 to prevent progression of ARDS, multiple organ failure, and death.

TP508 Reverses Lethal Effects of ARS. Animal studies show that a single injection of TP508 24 hours after lethal doses of whole-body radiation reverses effects of ARS to significantly increase survival and prevent delayed effects of ARS (DEARE) and loss of function in multiple organs including the lungs. In all tissues, radiation damages the vascular system causing endothelial dysfunction and endothelial cell apoptosis. TP508 reverses the radiation-induced endothelial dysfunction and accelerates DNA repair to restore vascular function and reduce coagulopathies that cause hemorrhage and micro thrombus formation in multiple organs. Recent studies in irradiated minipigs showed that a single injection of TP508 increased survival and reduced the amount of vascular hemorrhage in multiple organs including brains, hearts and lungs. Thus, it is envisioned by the inventors that administration of TP508 will prevent viral and cytokine-induced endothelial damage not only in the lungs, but throughout the body to have life-saving effects on those with severe COVID-19.

E. TP508 Efficacy Studies in Models of Lung Injury.

Two TP508 studies in animal models of respiratory injury have been conducted by Lovelace Respiratory Research Institute. In both studies, TP508 was administered as an inhaled aerosol prior to lung injury. One model was a mouse cigarette smoke inhalation model, the other was an acute lung injury model in which lung damage is caused in F344 rats by LPS inhalation. In both models, the lung insult disrupts the integrity of alveolar epithelial and endothelial cells, causing influx of inflammatory cells through the alveoli and a cytokine storm similar to the effects of SARS-CoV-2 infection in the lower lung.

TP508 Reduces Effects of Cigarette Smoke. In the mouse cigarette smoke model, aerosolized TP508 inhalation reduced cytokine expression by itself (IL-2, IL-5, IFN, IL-13, GM-CSF, KC, and IP-10), and had a dose dependent effect on cigarette smoke upregulation of certain cytokines, most predominantly GM-CSF (FIG. 6). As shown in FIG. 6, TP508 treatment reduced cigarette smoke (CS) induced upregulation of GM-CSF, but also decreased levels below that observed in animals breathing only filtered air (FA)

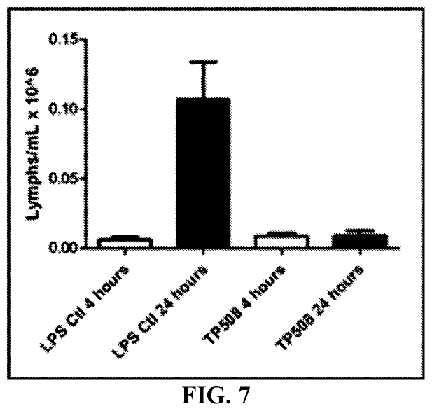

TP508 Reduces LPS-Induced Inflammation and Vascular Damage in Lung. In the F344 Rat LPS inflammation model, aerosolized TP508 inhalation prior to LPS significantly reduced LPS-induced expression of major cytokines (IL-1α, IL-1β, IL-2, IL-4, IL-6, IL-12, RANTES, and TNF-α) at 4 hours post LPS exposure. In addition, examination of cellular accumulation in bronchoalveolar lavage fluid (BALF) showed that TP508 pretreatment decreased the number of total lymphocytes that infiltrate by 24 hours into the lungs through capillary endothelial cells in the alveoli (FIG. 7).

These studies demonstrate that TP508 reduces lung injury related inflammation and decreases the loss of endothelial barrier function to reduce vascular leakage and cellular infiltration into alveolar spaces. Based on the inventors' knowledge of TP508 targeting of endothelial cells and restoration of vascular function, it is contemplated that systemic injection of TP508 will be even more effective in reversing effects of ARDS-related lung injury caused by SARS infections.

1. A method of treating a subject with acute respiratory distress or a subject at risk of developing acute respiratory distress comprising administering to the subject TP508 or a derivate thereof.

2. The method of claim 1, wherein the formulation is administered to the subject by injection.

3. The method of claim 1, wherein the formulation is administered to the subject by intravenous (IV) injection.

4. The method of claim 1, wherein the formulation is administered to the subject by subcutaneous injection.

5. The method of claim 1, wherein the formulation is administered to the subject by inhalation or instillation.

6. The method of claim 1, wherein the subject is diagnosed with a viral infection.

7. The method of claim 1, wherein the subject is diagnosed with a respiratory virus infection.

8. The method of claim 1, wherein the subject is diagnosed with a coronavirus or influenza infection.

9. The method of claim 1, wherein the subject is diagnosed with a SARS-CoV-2 infection.

10. A method for ameliorating post-respiratory virus infection syndrome comprising administering to the subject TP508 or a derivate thereof at least 5 to 15 days after infection by a respiratory virus.

11. The method of claim 10, wherein the formulation is administered to the subject by injection.

12. The method of claim 10, wherein the formulation is administered to the subject by intravenous (IV) injection.

13. The method of claim 10, wherein the formulation is administered to the subject by subcutaneous injection.

14. The method of claim 10, wherein the formulation is administered to the subject by inhalation or instillation.

15. The method of claim 10, wherein the respiratory virus infection is a coronavirus or influenza infection.

16. The method of claim 10, wherein the coronavirus infection is a SARS-CoV-2 infection.


SEQUENCE LISTING (23 sequences)

SEQ ID NO: 1 - protein, 23 aa, Artificial Sequence
  synthetic peptide
AGYKPDEGKRGDACEGDSGGPFV

SEQ ID NO: 2 - protein, 10 aa, Artificial Sequence
  synthetic peptide
CEGDSGGPFV

SEQ ID NO: 3 - protein, 10 aa, Artificial Sequence
  synthetic peptide
  MISC_FEATURE (2)..(2): X is Glu or Gln
  MISC_FEATURE (9)..(9): X is Phe, Met, Leu, His or Val
CXGDSGGPXV

SEQ ID NO: 4 - protein, 14 aa, Artificial Sequence
  synthetic peptide
  MISC_FEATURE (6)..(6): X is Glu or Gln
  MISC_FEATURE (13)..(13): X is Phe, Met, Leu, His or Val
RGDACXGDSGGPXV

SEQ ID NO: 5 - protein, 23 aa, Artificial Sequence
  synthetic peptide
AGYKPDEGKRGDACEGDSGGPFV

SEQ ID NO: 6 - protein, 14 aa, Artificial Sequence
  synthetic peptide
RGDACEGDSGGPFV

SEQ ID NO: 7 - protein, 33 aa, Artificial Sequence
  synthetic peptide
DNMFCAGYKPDEGKRGDACEGDSGGPFVMKSPF

SEQ ID NO: 8 - protein, 23 aa, Artificial Sequence
  synthetic peptide
  MISC_FEATURE (15)..(15): X is Glu or Gln
  MISC_FEATURE (22)..(22): X is Phe, Met, Leu, His or Val
AGYKPDEGKRGDACXGDSGGPXV

SEQ ID NO: 9 - protein, 33 aa, Artificial Sequence
  synthetic peptide
  MISC_FEATURE (20)..(20): X is Glu or Gln
  MISC_FEATURE (27)..(27): X is Phe, Met, Leu, His or Val
DNMFCAGYKPDEGKRGDACXGDSGGPXVMKSPF

SEQ ID NO: 10 - protein, 14 aa, Artificial Sequence
  synthetic peptide
  MISC_FEATURE (5)..(5): X is Ala, Gly, Ser or an S-protected Cysteine
  MISC_FEATURE (6)..(6): X is Glu or Gln
  MISC_FEATURE (13)..(13): X is Phe, Met, Leu, His or Val
RGDAXXGDSGGPXV

SEQ ID NO: 11 - protein, 23 aa, Artificial Sequence
  synthetic peptide
  MISC_FEATURE (14)..(14): X is Ala, Gly, Ser or an S-protected cysteine.
AGYKPDEGKRGDAXEGDSGGPFV

SEQ ID NO: 12 - protein, 23 aa, Artificial Sequence
  synthetic peptide
  MISC_FEATURE (14)..(14): X is Ala, Gly, Ser or an S-protected cysteine.
  MISC_FEATURE (15)..(15): X is Glu or Gln
  MISC_FEATURE (22)..(22): X is Phe, Met, Leu, His or Val
AGYKPDEGKRGDAXXGDSGGPXV

SEQ ID NO: 13 - protein, 23 aa, Artificial Sequence
  synthetic peptide
AGYKPDEGKRGDAAEGDSGGPFV

SEQ ID NO: 14 - protein, 23 aa, Artificial Sequence
  synthetic peptide
AGYKPDEGKRGDAAEGDSGGPFV

SEQ ID NO: 15 - protein, 23 aa, Artificial Sequence
  synthetic peptide
AGYKPDEGKRGDASEGDSGGPFV

SEQ ID NO: 16 - protein, 33 aa, Artificial Sequence
  synthetic peptide
  MISC_FEATURE (5)..(5): X is Ala, Gly, Ser, or an S-protected cysteine
  MISC_FEATURE (19)..(19): X is Ala, Gly, Ser, or an S-protected cysteine
DNMFXAGYKPDEGKRGDAXEGDSGGPFVMKSPF

SEQ ID NO: 17 - protein, 33 aa, Artificial Sequence
  synthetic peptide
  MISC_FEATURE (5)..(5): X is Ala, Gly, Ser, or an S-protected cysteine
  MISC_FEATURE (19)..(19): X is Ala, Gly, Ser, or an S-protected cysteine
  MISC_FEATURE (20)..(20): X is Glu or Gln
  MISC_FEATURE (27)..(27): X is Phe, Met, Leu, His or Val
DNMFXAGYKPDEGKRGDAXXGDSGGPXVMKSPF

SEQ ID NO: 18 - protein, 33 aa, Artificial Sequence
  synthetic peptide
DNMFAAGYKPDEGKRGDAAEGDSGGPFVMKSPF

SEQ ID NO: 19 - protein, 33 aa, Artificial Sequence; identical to SEQ ID NO: 18
  synthetic peptide

SEQ ID NO: 20 - protein, 33 aa, Artificial Sequence
  synthetic peptide
AGYKPDEGKRGDACEGDSGGPFVMKSPFNNRWY

SEQ ID NO: 21 - protein, 33 aa, Artificial Sequence
  synthetic peptide
  MISC_FEATURE (15)..(15): X is Glu or Gln
  MISC_FEATURE (22)..(22): X is Phe, Met, Leu, His or Val
AGYKPDEGKRGDACXGDSGGPXVMKSPFNNRWY

SEQ ID NO: 22 - protein, 33 aa, Artificial Sequence; identical to SEQ ID NO: 20
  synthetic peptide

SEQ ID NO: 23 - protein, 33 aa, Artificial Sequence; identical to SEQ ID NO: 20
  synthetic peptide